(19) United States
(12) Patent Application Publication
(10) Pub. No.: US 2024/0382090 A1
(43) Pub. Date: Nov. 21, 2024

(54) OPHTHALMIC IMAGING SYSTEM

(71) Applicant: YOUNG OPTICS INC., Hsinchu Science Park (TW)

(72) Inventors: Ching-Lun LIN, Hsinchu Science Park (TW); Ching-Sheng CHANG, Hsinchu Science Park (TW); Cheng-Hung CHEN, Hsinchu Science Park (TW)

(21) Appl. No.: 18/621,857

(22) Filed: Mar. 29, 2024

(30) Foreign Application Priority Data
May 18, 2023 (TW) 112118547
Feb. 5, 2024 (TW) 113104518

(51) Int. Cl. A61B 3/12; A61B 3/14

(52) U.S. Cl. CPC A61B 3/12; A61B 3/14

(57) ABSTRACT

An ophthalmic imaging system includes a first lens group, an illumination light source, an imaging module, a fixation light, a reflective element, at least one light-blocking member, and a first polarizer. The first lens group includes an aspheric first lens. The fixation light provides a light beam and includes at least three light-emitting components. The reflective element is configured to reflect the light beam and direct the light beam towards the aspheric first lens. The light-blocking member is disposed downstream from and in a light path of the fixation light and disposed upstream from and in a light path of the reflective element. The first polarizer is disposed downstream from and in the light path of the fixation light and disposed upstream from and in the light path of the reflective element to polarize the light beam.

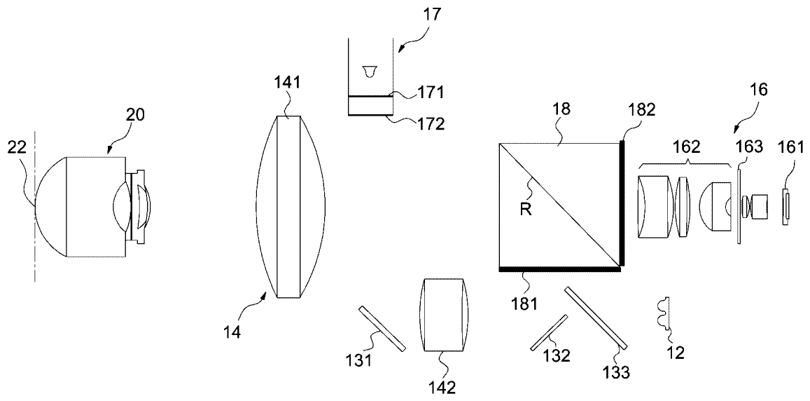

### CROSS-REFERENCE TO RELATED APPLICATION

This application claims the priority benefit of Taiwan application serial no. 112118547, filed May 18, 2023, and Taiwan application serial no. 113104518, filed Feb. 5, 2024. The entirety of the above-mentioned patent applications is hereby incorporated by reference herein and made a part of this specification.

### BACKGROUND

### Field of the Invention

The invention relates to an imaging system, more particularly to an ophthalmic imaging system.

### Description of the Related Art

Typically, fundus photography requires an illumination light source powerful enough to illuminate the fundus. The illumination light sources traditionally used for fundus photography are often placed at a position far from the eyepiece (the lens closest to the eyeball), resulting in poor illumination efficiency. Therefore, the integration of extra optical components is required to improve the illumination efficiency.

### BRIEF SUMMARY OF THE INVENTION

In order to achieve one or a portion of or all of the objects or other objects, one embodiment of the invention provides an ophthalmic imaging system including a first lens group having a positive refractive power, an illumination light source, an imaging module, a fixation light, a reflective element, at least one light-blocking member, and a first polarizer. The first lens group includes an aspheric first lens, and the illumination light source is capable of forming an illumination zone between the illumination light source and the first lens group. The imaging module is capable of forming an imaging zone between the imaging module and the first lens group, and the imaging zone covers an optical axis of the first lens group. The fixation light provides a light beam and includes at least three light-emitting components. The reflective element is disposed downstream from and in a light path of the fixation light and disposed upstream from and in a light path of the aspheric first lens. The light-blocking member is disposed downstream from and in the light path of the fixation light and disposed upstream from and in a light path of the reflective element. The first polarizer is disposed downstream from and in the light path of the fixation light and disposed upstream from and in the light path of the reflective element to polarize the light beam.

Another embodiment of the invention provides an ophthalmic imaging system including a first lens group having a positive refractive power, an illumination light source, an imaging module, a fixation light, a reflective element, at least one light-blocking member, and a first polarizer. The first lens group includes an aspheric first lens, and the imaging module is disposed on an optical axis of the first lens group. The fixation light provides a light beam and includes at least three light-emitting components. The reflective element is configured to reflect the light beam and direct the light beam towards the aspheric first lens. The light-blocking member is disposed downstream from and in a light path of the fixation light and disposed upstream from and in a light path of the reflective element. The first polarizer is disposed downstream from and in the light path of the fixation light and disposed upstream from and in the light path of the reflective element to polarize the light beam.

According to the above embodiments, by setting the positional relationships among the illumination light source, the first lens group, the fixation light, the reflective element and the fold mirrors, the components of the entire system can be arranged more compactly within a limited barrel space to facilitate the miniaturization of an ophthalmic imaging system. Moreover, by placing the first and second light-blocking members in a light path between the fixation light and the reflective element, the position and angle of the light beams emitted by the fixation light can be restricted to reduce aberrations in the image beams projected onto the fundus. Furthermore, a first polarizer and a second polarizer are provided to polarize the light beam emitted by the fixation light before entering the reflective element and to polarize the light beam reflected by the fundus towards the imaging module. The polarization directions of the first polarizer and the second polarizer are different to prevent light beams emitted by the illumination light source or the fixation light from directly entering the imaging module, thus reducing stray light and improving the imaging quality of the ophthalmic imaging system.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The sole FIGURE shows a schematic diagram of an ophthalmic imaging system according to an embodiment of the invention.

### DETAILED DESCRIPTION OF THE INVENTION

In the following detailed description of the preferred embodiments, directional terminology, such as “top,” “bottom,” “front,” “back,” etc., is used with reference to the orientation of the FIGURE(s) being described. The components of the invention can be positioned in a number of different orientations. As such, the directional terminology is used for purposes of illustration and is in no way limiting. Further, “First,” “Second,” etc., as used herein, are used as labels for nouns that they precede, and do not imply any type of ordering (e.g., spatial, temporal, logical, etc.).

The sole FIGURE shows a schematic diagram of an ophthalmic imaging system according to an embodiment of the invention. As shown in the sole FIGURE, the ophthalmic imaging system 10 includes an illumination light source 12, a first lens group (eyepiece group) 14, an imaging module 16, a fixation light 17, a reflective element 18, a first light-blocking member 171 and a second light-blocking member 172. Specifically, the illumination light source 12 may include an infrared light source and a white light source, and the infrared or white light source can be, for example, a light-emitting diode (LED), an organic light-emitting diode (OLED), or a laser diode (LD). The first lens group 14 has a positive refractive power and at least includes an aspheric lens 141, and the aspheric lens 141 may be a molded-glass lens. The illumination light source 12 is capable of forming an illumination zone between the illumination light source 12 and the first lens group 14, the imaging module 16 is capable of forming an imaging zone between the imaging module 16 and the first lens group 14, and the imaging zone covers an optical axis of the first lens group 14. In this embodiment, the molded-glass aspheric lens 141 is the closest lens to the object side (closest to the eyeball 20) within the first lens group 14 and has a non-zero refractive power. Specifically, the reflective element 18 may be a polarizing beam splitter (PBS) having a reflective surface R. The reflective surface R may direct light beams traveling through the imaging and illumination paths towards the first lens group 14, and then the light beams may pass through the first lens group 14 to reach the fundus (retina) 22 of an individual being examined (a subject), thereby enabling illumination and image pick-up functions of the ophthalmic imaging system 10. In one embodiment, the first light-blocking member 171 and the second light-blocking member 172 are aperture stops, the fixation light 17 may have at least three light-emitting components, such as three light-emitting diodes (LEDs), and the three light-emitting components may each produce its own light and collectively provide a light beam. The fixation light 17 can keep the subject's eye fixed at a specific observation angle to prevent involuntary eye movements. The three differently positioned light-emitting components such as LEDs can fix the subject's eye at a certain angle based on the particular requirements. Moreover, the light-blocking members 171 and 172 can be disposed downstream from and in a light path of the fixation light 17 and disposed upstream from and in a light path of the reflective element 18. Such arrangement is allowed to restrict the position and angle of the light beam emitted by the fixation light 17 to reduce aberrations in the image beams of the fixation light 17 projected onto the fundus.

In this embodiment, the ophthalmic imaging system 10 also includes a first fold mirror 131, a second fold mirror 132, a third fold mirror 133, a relay lens 142, a first polarizer 181, and a second polarizer 182. The first fold mirror 131 is disposed downstream from and in a light path of the fixation light 17 and disposed upstream from and in a light path of the reflective element 18 to direct the light beam emitted by the fixation light 17 towards the reflective element 18. The second fold mirror 132 is disposed downstream from and in a light path of the fixation light 17 and the first fold mirror 131 and disposed upstream from and in the light path the reflective element 18. The relay lens 142 is disposed in a light path between the first fold mirror 131 and the second fold mirror 132. The third fold mirror 133 is disposed downstream from and in a light path of the illumination light source 12 and disposed upstream from and in a light path of the reflective element 18. The first polarizer 181 is disposed downstream from and in a light path of the fixation light 17, the first fold mirror 131 and the second fold mirror 132. The second polarizer 182 is disposed downstream from and in a light path of the reflective element 18 and disposed upstream from and in a light path of the imaging module 16 to polarize the light beam from the illumination light source 12. Specifically, a light beam emitted from the fixation light 17 is guided by the first fold mirror 131, the relay lens 142 and the second fold mirror 132 towards the reflective element 18, and then the light beam is deflected by the reflective element 18 to pass through the first lens group 14 to form an image on the fundus 22 of the subject. Furthermore, the infrared and/or white light beam emitted by the illumination light source 12 is reflected by the third fold mirror 133 towards the reflective element 18, and then reflected by the reflective element 18 to pass through the first lens group 14 to illuminate the fundus 22 of the subject.

In this embodiment, because the first polarizer 181 is disposed downstream of the fixation light 17 and upstream of the reflective element 18 along the light path, the light beam from the fixation light 17 is polarized prior to reaching the reflective element 18. This arrangement may reduce stray light within the imaging zone, thereby enhancing the image quality. Similarly, the second polarizer 182 is disposed downstream of the reflective element 18 and upstream of the imaging module 16 along the light path to polarize the light beam that is reflected by the fundus 22 of the eyeball 20 towards the imaging module 16. The polarization directions of the first polarizer 181 and the second polarizer 182 are different to prevent light beams emitted by the illumination light source 12 or the fixation light 17 from directly entering the imaging module 16 through the reflective element 18, with these undesired light beams entering the imaging module 16 contributing to stray light, and therefore the image quality of the ophthalmic imaging system 10 can be improved.

In this embodiment, the imaging module 16 includes an image sensor 161 and a second lens group 162. The second lens group 162 may include multiple lenses with refractive powers or a singlet lens with a refractive power. The imaging module 16 may further include an infrared light filter 163 disposed in a light path between the image sensor 161 and the second lens group 162. The fundus 22 reflects the illumination light beam to form an image beam, and the image beam sequentially passes through the first lens group (eyepiece group) 14 and the second lens group 162 and is then focused onto the image sensor 161 of the imaging module 16, thereby forming a fundus image. The ophthalmic imaging system 10 may further include a display (not shown) to display the fundus photograph output by the image sensor 161.

Based on the design of various embodiments of the invention, by setting the positional relationships among the illumination light source, the first lens group, the fixation light, the reflective element and the fold mirrors, the components of the entire system can be arranged more compactly within a limited barrel space to facilitate the miniaturization of an ophthalmic imaging system. Moreover, by placing the first and second light-blocking members in a light path between the fixation light and the reflective element, the position and angle of the light beams emitted by the fixation light can be restricted to reduce aberrations in the image beams projected onto the fundus. Furthermore, a first polarizer and a second polarizer are provided to polarize the light beam emitted by the fixation light before entering the reflective element and to polarize the light beam reflected by the fundus towards the imaging module. The polarization directions of the first polarizer and the second polarizer are different to prevent light beams emitted by the illumination light source or the fixation light from directly entering the imaging module, thus reducing stray light and improving the imaging quality of the ophthalmic imaging system.

Though the embodiments of the invention have been presented for purposes of illustration and description, they are not intended to be exhaustive or to limit the invention. Accordingly, many modifications and variations without departing from the spirit of the invention or essential characteristics thereof will be apparent to practitioners skilled in this art. It is intended that the scope of the invention be defined by the claims appended hereto and their equivalents in which all terms are meant in their broadest reasonable sense unless otherwise indicated.

What is claimed is:

1. An ophthalmic imaging system, comprising:
a first lens group having a positive refractive power and comprising an aspheric first lens;
an illumination light source capable of forming an illumination zone between the illumination light source and the first lens group;
an imaging module capable of forming an imaging zone between the imaging module and the first lens group, and the imaging zone covering an optical axis of the first lens group;
a fixation light for providing a light beam, wherein the fixation light comprises at least three light-emitting components;
a reflective element disposed downstream from and in a light path of the fixation light and disposed upstream from and in a light path of the aspheric first lens;
at least one light-blocking member disposed downstream from and in the light path of the fixation light and disposed upstream from and in a light path of the reflective element; and
a first polarizer disposed downstream from and in the light path of the fixation light and disposed upstream from and in the light path of the reflective element to polarize the light beam.

2. The ophthalmic imaging system as claimed in claim 1, wherein the aspheric first lens is a glass-molded lens, and the at least one light-blocking member is an aperture stop.

3. The ophthalmic imaging system as claimed in claim 1, wherein the reflective element is a polarizing beam splitter having a reflective surface, and the reflective surface is capable of directing the light beam onto an eyeball to illuminate a fundus of the eyeball.

4. The ophthalmic imaging system as claimed in claim 1, further comprising a first fold mirror disposed downstream from and in the light path of the fixation light and disposed upstream from and in the light path of the reflective element to reflect and direct the light beam towards the reflective element.

5. The ophthalmic imaging system as claimed in claim 4, further comprising a second fold mirror disposed downstream from and in the light path of the fixation light, downstream from and in a light path of the first fold mirror, and upstream from and in the light path of the reflective element.

6. The ophthalmic imaging system as claimed in claim 5, further comprising a relay lens disposed in a light path between the first fold mirror and the second fold mirror.

7. The ophthalmic imaging system as claimed in claim 5, further comprising a third fold mirror disposed downstream from and in a light path of the illumination light source and disposed upstream from and in a light path of the reflective element.

8. The ophthalmic imaging system as claimed in claim 5, wherein the first polarizing element is disposed downstream from and in a light path of the fixation light, the first fold mirror and the second fold mirror.

9. The ophthalmic imaging system as claimed in claim 1, further comprising a second polarizer disposed downstream from and in a light path of the reflective element and disposed upstream from and in a light path of the imaging module to polarize the light beam from the illumination light source.

10. The ophthalmic imaging system as claimed in claim 1, wherein the imaging module includes a second lens group, an infrared light filter and an image sensor, and the infrared light filter is disposed in a light path between the second lens group and the image sensor.

11. An ophthalmic imaging system, comprising:
a first lens group having a positive refractive power and comprising an aspheric first lens;
an illumination light source;
an imaging module disposed on an optical axis of the first lens group;
a fixation light for providing a light beam, wherein the fixation light comprises at least three light-emitting components;
a reflective element configured to reflect the light beam and direct the light beam towards the aspheric first lens;
at least one light-blocking member disposed downstream from and in a light path of the fixation light and disposed upstream from and in a light path of the reflective element; and
a first polarizer disposed downstream from and in the light path of the fixation light and disposed upstream from and in the light path of the reflective element to polarize the light beam.

12. The ophthalmic imaging system as claimed in claim 11, wherein the aspheric first lens is a glass-molded lens, and the at least one light-blocking member is an aperture stop.

13. The ophthalmic imaging system as claimed in claim 11, wherein the reflective element is a polarizing beam splitter having a reflective surface, and the reflective surface is capable of directing the light beam onto an eyeball to illuminate a fundus of the eyeball.

14. The ophthalmic imaging system as claimed in claim 11, further comprising a first fold mirror disposed downstream from and in the light path of the fixation light and disposed upstream from and in the light path of the reflective element to reflect and direct the light beam towards the reflective element.

15. The ophthalmic imaging system as claimed in claim 14, further comprising a second fold mirror disposed downstream from and in the light path of the fixation light, downstream from and in a light path of the first fold mirror, and upstream from and in the light path of the reflective element.

16. The ophthalmic imaging system as claimed in claim 15, further comprising a relay lens disposed in a light path between the first fold mirror and the second fold mirror.

17. The ophthalmic imaging system as claimed in claim 15, further comprising a third fold mirror disposed downstream from and in a light path of the illumination light source and disposed upstream from and in a light path of the reflective element.

18. The ophthalmic imaging system as claimed in claim 15, wherein the first polarizing element is disposed downstream from and in a light path of the fixation light, the first fold mirror and the second fold mirror.

19. The ophthalmic imaging system as claimed in claim 11, further comprising a second polarizer disposed downstream from and in a light path of the reflective element and disposed upstream from and in a light path of the imaging module to polarize the light beam from the illumination light source.

20. The ophthalmic imaging system as claimed in claim 11, wherein the imaging module includes a second lens group, an infrared light filter and an image sensor, and the infrared light filter is disposed in a light path between the second lens group and the image sensor.
